(19) United States
(12) Patent Application Publication
(10) Pub. No.: US 2023/0172828 A1
(43) Pub. Date: Jun. 8, 2023

(54) ANTI-AGING DERMATOLOGICAL COMPOSITIONS

(71) Applicant: Dr. Sabrina Ltd., Wiltshire (GB)

(72) Inventors: Sabrina Shah-Desai, Wiltshire (GB); Chloe Pressoir de Valcy, London (GB)

(21) Appl. No.: 18/076,001

(22) Filed: Dec. 6, 2022

(30) Foreign Application Priority Data
Dec. 7, 2021 (GB) 2117621.9

(51) Int. Cl. A61K 8/49; A61K 8/60; A61K 8/41; A61K 8/34; A61K 8/92; A61K 8/73; A61K 8/365; A61K 8/99; A61K 8/64; A61K 8/9789; A61K 8/9794; A61K 8/9722; A61Q 19/02; A61Q 19/08

(52) U.S. Cl. CPC A61K 8/4926; A61K 8/34; A61K 8/41; A61K 8/64; A61K 8/73; A61K 8/99; A61K 8/345; A61K 8/347; A61K 8/365; A61K 8/602; A61K 8/922; A61K 8/4953; A61K 8/9722; A61K 8/9789; A61K 8/9794; A61Q 19/02; A61Q 19/08

(57) ABSTRACT

The invention relates to multi-action dermatological compositions that provides lightening, protecting and anti-aging effects to the skin of a user. The composition comprises niacinamide, arbutin, tranexamic acid, ubiquinone, a DHEA-like component, curcuma oily extract and marine exopolysaccharides. The invention also relates to methods of dermatological treatment and a method of treating hyperpigmentation by applying the composition to the skin of a user.

### CROSS REFERENCE TO RELATED APPLICATIONS

The application claims priority under 35 U.S.C. §119 to Great Britain Application No. 2117621.9, filed Dec. 7, 2021, the disclosure of which is incorporated herein by reference.

### FIELD OF THE INVENTION

The invention relates to multi-action dermatological compositions that provide lightening, restructuring, anti-oxidising, lifting and anti-aging effects to the skin of a user. The invention also relates to methods of dermatological treatment by applying the composition to the skin of a user.

### BACKGROUND TO THE INVENTION

Skin is particularly susceptible to damage by environmental stresses. Ultraviolet (UV) radiation, smoking and air pollution increase oxidative activity in the skin, damaging cellular and extracellular components. Furthermore, environmental stresses may deplete the moisture of the skin, exacerbating the appearance of fine lines and wrinkles.

Collagen and hyaluronic acid levels in the skin also decline with age, which results in a loss of moisture, strength and elasticity of the skin. As a result, characteristic signs of aging emerge such as fine lines and wrinkles, sagging skin and an uneven complexion.

UV light exposure in the presence of oxygen results in the creation of free radicals in the skin. These radicals frequently trigger the release of inflammatory mediators, commonly resulting in sun burn, cytoskeletal alterations, the depletion of collagen, and structural DNA damage. (Poljsak and Dahmane, “Free Radicals and Extrinsic Skin Aging”, Dermatology Research and Practice 2012, 1-4 (2012); Weringhaus et al., “Evaluation of the photoprotective effect of oral vitamin E supplementation”, Archives of Dermatology 130(10), 1257-1261 (1994)). The body neutralises the free radicals caused by UV light with antioxidants. Natural enzymatic antioxidants used by the body include superoxide dismutase, catalase and glutathione peroxidase; while known non-enzymatic antioxidants that may assist the skin in scavenging UV induced free radicals include vitamin A, vitamin C and vitamin E (Nimse and Pal, “Free radicals, natural antioxidants, and their reaction mechanisms” RSC Advances 5(35), 27986-28006 (2015)).

Exposure of the skin to UV rays triggers the production of melanin by melanocytes. Extra melanin causes the skin to appear darker, and in some cases, the sun causes an uneven increase in melanin production, resulting in irregular pigmentation. Hyperpigmentation, which is caused by an excess production of melanin can also be caused by sun damage and other skin injuries such as acne vulgaris.

Melanocyte distribution becomes less regulated by the body with age, and when UV exposure stimulates melanocyte activity, hyperpigmentation occurs where the concentration of the cells is greatest. As a result, dark spots or age spots may emerge.

Hyperpigmentation is also associated with a number of conditions including Addison’s disease, Cushing’s disease, acanthosis nigricans, melasma, acne scarring, linea nigra, Peutz-Jeghers syndrome, smoker’s melanosis, coeliac disease, Cronkhite-Canada syndrome, porphyria, tinea fungal infections such as ringworm, haemochromatosis, aromatase deficiency, Nelson’s syndrome, Grave’s disease, Schimke immunoosseous dysplasia and tinea cruris. In addition, hyperpigmentation may also be the result of exposure to certain chemicals such as salicylic acid, bleomycin, cisplatin or mercury.

Skin whitening agents are often used to treat hyperpigmentation and melasma by reducing the content of melanin in the skin. Tyrosinase is an enzyme present in melanosomes that catalyses the production of melanin from tyrosine by oxidation. Inhibition of the activity of tyrosinase, prevention of the expression or activation of tyrosinase and therefore known to lighten the skin.

There remains a need to provide improved multi-action dermatological compositions that provide skin lightening effects to combat hyperpigmentation, protect the skin from environmental factors, while minimising the signs of aging by reducing the appearance of fine lines and wrinkles.

### SUMMARY OF THE INVENTION

The inventors have developed a multi-action dermatological composition known as the Kiara molecule complex. This anti-aging whitening complex imparts a lightening, global restructuring, anti-oxidising and lifting effect when applied topically to the skin of a user.

In a first aspect, the invention relates to the Kiara molecule complex which is a dermatological composition comprising niacinamide, arbutin, tranexamic acid, ubiquinone, a DHEA-like component, curcuma oily extract and marine exopolysaccharides. Optionally, the composition comprises ascorbyl glucoside.

In a second aspect, the invention relates to a method of dermatological treatment using the Kiara molecule complex. A dermatological composition is provided comprising niacinamide, arbutin, tranexamic acid, ubiquinone, a DHEA-like component, curcuma oily extract and marine exopolysaccharides. Optionally, the composition comprises ascorbyl glucoside. The composition is then topically administered to the skin of a user.

In a third aspect, the invention relates to a method of treating hyperpigmentation using the Kiara molecule complex.

### BRIEF DESCRIPTION OF THE DRAWINGS

FIG. 1 outlines the mechanism of melanogenesis. Tyrosine in melanocytes is converted to L-DOPA then DOPAquinone by the enzyme tyrosinase. If cysteine is present, DOPAquinone is converted to cysteinylDOPA which undergoes oxidation polymerisation to form pheomelanin, which is a yellow to red colour. In the absence of cysteins, DOPAquinone may be catalysed by tyrosine related protein-2 (TRP2) to DHICA, which then may be catalysed by tyrosine related protein-1 (TRP1) to DHICA-melanin. Alternatively, DOPAchrome may be converted in a non-enzymatic reaction to DHI, which then produces DHI-melanin. Both DHICA-melanin an DHI-melanin are eumelanins which are brown to black in colour.

### DETAILED DESCRIPTION

The inventor has developed the Kiara molecule complex, which is a dermatological composition that provides a multitude of skin enhancing effects when applied topically to the skin of a user.

Specifically, the Kiara molecule complex imparts a skin lightening, global restructuring, anti-oxidising effect and lifting effect. Combined, these effects create an overall anti-aging impact, altering the pigmentation of the skin and improving its strength and texture.

The Kiara molecule complex comprises niacinamide, arbutin, tranexamic acid, ubiquinone, a DHEA-like component, curcuma oily extract and marine exopolysaccharides. Optionally, the Kiara molecule complex also comprises ascorbyl glucoside.

The skin lightening effect of the Kiara molecule is achieved through the synergistic action of the active ingredients niacinamide, tranexamic acid, arbutin, the curcuma oily extract and the optional ascorbyl glucoside; the global restructuring and anti-oxidising effects are imparted by the combined use of tranexamic acid, ubiquinone, curcuma oily extract DHEA-like component, and the optional ascorbyl glucoside; and the instant filling and lifting effect is achieved through the use of marine exopolysaccharides. The Kiara molecule therefore not only combats hyperpigmentation and dark spots by lightening, and whitening the skin but also restructures, lifts and protects the skin, resulting in an effective anti-aging product that leaves the skin bright, strong and rejuvenated.

Melanin is a natural pigment produced by melanocyte cells in the bottom layer of the skin’s epidermis. Different types of melanin may be produced by the skin -eumelanin, which is a heterogenous brown to black colour and exhibits antioxidant properties; and pheomelanin, which is red to yellow in colour, is responsible for freckles, and exhibits phototoxic pro-oxidant action.

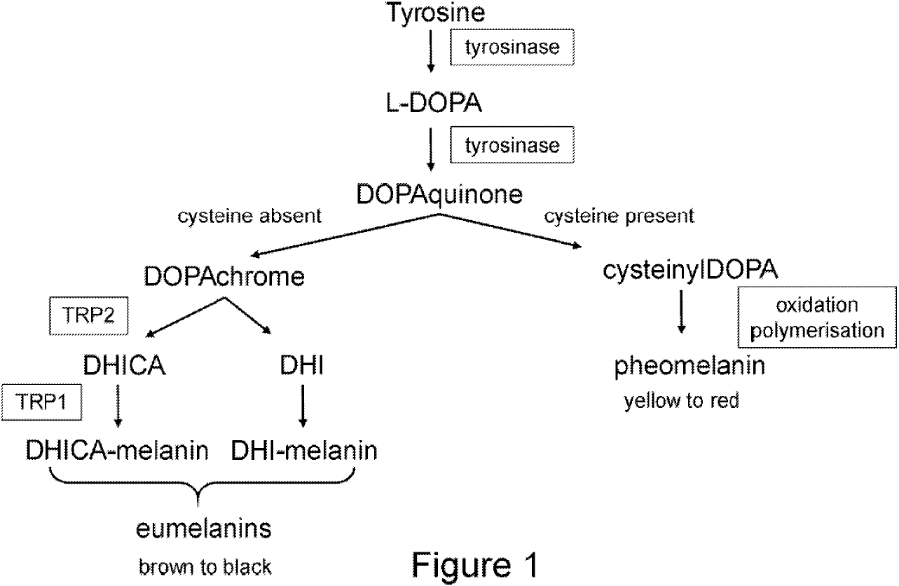

The mechanism of melanin formation in melanocytes is known as melanogenesis. This process leads to long lasting pigmentation, and is distinct from the pigmentation that originates from oxidation of pre-existing melanin. The basic mechanism of melanogenesis can be explained with reference to FIG. 1. The enzyme tyrosinase catalyses the hydroxylation of tyrosine to 3,4-dihydroxyphenylalanine (L-DOPA), and the oxidation of L-DOPA to DOPAquinone. The type of melanin produced is dependent on the availability of substrates and the function of melanogenetic enzymes. In the presence of cysteine, DOPAquinone may be converted to 3- or 5-cysteinylDOPA which subsequently undergoes oxidation polymerisation to form yellow to red pheomelanin.

An absence of cysteine causes DOPAquinone to undergo rapid, spontaneous intramolecular cyclization and further rearrangement to form DOPAchrome. From here, DOPAchrome may form eumelanin by two distinct pathways. The first pathway involves tyrosine related protein-2 (TRP2) to catalyse the tautomerisation of DOPAchrome to form DHI-carboxylic acid (DHICA). Then, tyrosine related protein-1 (TRP1) may catalyse the oxidation of DHICA to form DHICA-melanin, which is typically light brown in colour. In the second pathway, DOPAchrome undergoes spontaneous decarboxylation, forming 5,6-dihydroxyindole (DHI). DHI then rapidly oxidises and polymerises to form DHI-melanin, which is typically dark brown or black in colour (Cichorek et al., “Heterogeneity of neural crest-derived melanocytes”, Central European Journal of Biology 8(4), 315-330 (2013)).

After melanin synthesis, melanin is contained in organelles called melanosomes. The melanosomes can be transported from melanocytes to nearby keratinocytes, which are the primary cell type found in the epidermis. This process induces pigmentation in the outermost layer of the skin.

The Kiara molecule complex has a lightening effect through the action of active ingredients niacinamide, tranexamic acid, curcuma oily extract, arbutin and the optional ascorbyl glucoside.

Niacinamide, otherwise known as nicotinamide, is a form of vitamin B₃. The mechanism of skin whitening involves inhibiting melanosome transfer from melanocytes to keratinocytes. Niacinamide provides additional benefits to the skin, such as stimulating fibroblasts, reducing collagen breakdown, improving the metabolism of the skin and accelerating dead skin cell exfoliation.

Niacinamide may be present in the dermatological composition in the range of from 0.005 to 10 wt. % of the total composition, preferably from 0.01 to 8 wt. %, more preferably from 1 to 5 wt. %.

Tranexamic acid performs as a whitening agent and super-anti-oxidant. In inhibits tyrosinase synthesis in melanocytes in addition to blocking the transfer of pigment from melanocytes to keratinocytes in the epidermis.

Tranexaminc acid may be present in the dermatological composition in the range of 0.05 to 10 wt.% of the total composition, preferably from 0.1 to 5 wt.%, more preferably from 0.5 to 3 wt.%.

The curcuma oily extract may comprise curcuma longa root (turmeric) extract. It may further comprise helianthus annus (sunflower) seed oil. The curcuma oily extract preferably comprises around 10 wt.% curcuma longa root extract and around 90 wt.% helianthus annus (sunflower) seed oil. It efficiently protects the skin against the harmful effects of infrared (IR) radiation as well as blue light. By blocking IR and blue light-induced free-radical formation, it can effectively protect mitrochondrial DNA, inhibit light-induced skin aging, and prevent the loss of skin density upon sun exposure.

The curcuma oily extract may be present in the dermatological composition in the range of 0.001 to 5 wt.% of the total composition, preferably from 0.01 to 3 wt.%, more preferably from 0.01 to 1 wt.%.

The arbutin component of the Kiara molecule complex has been shown to have a dual effect on the lightening of skin. Firstly, it directly inhibits tyrosinase activity, and therefore prevents melanogenesis from occurring. Secondly, it inhibits the formation of the enzyme tyrosinase. Through this tyrosinase regulation, melanin formation and hyperpigmentation can be prevented, and the skin lightens as a result. The arbutin is a liposomal form, which optimises the effectiveness of the whitening components of the Kiara molecule complex.

Arbutin may be present in the dermatological composition in the range of from 0.001 to 5 wt. % of the total composition, preferably from 0.005 to 2 wt. %, more preferably from 0.1 to 1 wt. %.

Ascorbyl glucoside, a stable form of vitamin C combined with glucose, may optionally be included in the compositions of the invention to contribute to the skin lightening effect by inhibiting tyrosinase. As a non-enzymatic antioxidant, this component may also aid in the scavenging of free radicals cause by exposure of the skin to UV radiation.

The activity of arbutin in inhibiting tyrosinase activity may be reinforced by reduced glutathione and vitamin C, which have both whitening and antioxidant properties. Further, the liposomial form optimises the effectiveness of these components.

Global restructuring and anti-oxidising effects of the Kiara molecule complex are imparted by the combined use of tranexamic acid, ubiquinone, curcuma oily extract, DHEA-like component and the optional ascorbyl glucoside.Ubiquinone is also known as coenzyme Q10. The ubiquinone of the Kiara molecule may be produced by bacterial fermentation process or in a half synthetic process starting from tobacco plant and Solanesol extracted from the plant. This enzyme is also produced by the human body, performing as a lipophilic antioxidant and radical scavenger, consequently inhibiting lipid peroxidation, lipid and protein oxidation in skin cells and regenerating other antioxidants like tocopherol. In keratinocytes and fibroblasts, it protects against active oxygen structures like superoxide, singuletteoxygene and hydroxyl radicals which contribute to the process of photoaging of skin. Its dermal application therefore reduces the effects of environmental stress, resulting in the reduction of wrinkle depth and decreased turnover time of the epithelium.

Ubiquinone may be present in the dermatological composition of the invention in the range of from 0.001 to 5 wt. % of the total composition, preferably from 0.005 to 2 wt. %, more preferably from 0.05 to 1 wt. %.

An increase in age is accompanied by a decrease in the level of the hormone dehydroepiandrosterone (DHEA) and the subsequent doubling of the cytokine interleukin-6 (IL-6) in the skin. Furthermore, daily exposure to UV light results in an increase in the presence of inflammatory IL-6 in the skin. Disregulation of this cytokine is believed to play a key role in the remodelling of the immune system at older age, with evidence suggesting a reduced ability to control systemic inflammation which leads to signs of aging (Rea et al., “Age and Age-Related Diseases Role of Inflammation Triggers and Cytokines”, Frontiers in Immunology 9(586), 1-28 (2018)).

Use of DHEA-like component in the Kiara molecule complex allows specific intervention on these mechanisms by gradually reducing the overproduction of IL-6 in keratinocytes and fibroblasts, without noticeably affecting other cytokines. Its use results in stimulating the activities of endorphin and DHEA, and stimulates the biosynthesis of DHEA.

As a result, this composition improves isotropy, reduces roughness, reduces deep microgrooves and increase firmness, elasticity and moisture. The DHEA-like component may comprise glycerin, steareth-20, palmitoyl tetrapeptide-7, chlorohexidine digluconate and potassium sorbate. It may be water based. It may be derived from a concentrated extract of monk’s pepper berries. Preferably, the DHEA-like component has around 82 wt. % water, around 15 wt.% glycerin, 3 wt. % steareth-20, 0.2 wt. % chlorhexidine diglutonate, 0.1 wt.% potassium sorbate and 0.05 wt.% palmitoyl tetrapeptide-7.

Glycerin is a polyol, exhibits antimicrobial and antiviral properties and acts as a humectant. Accordingly, glycerin functions as an effective moisturising agent when applied to the skin, drawing up moisture through the layers of the skin and from the air.

In addition to the glycerine in the DHEA-like component, further amounts of glycerin may be added to the dermatological composition of the invention in the range of from 0.01 to 15 wt. % of the total composition, preferably from 0.1 to 12 wt. %, more preferably from 1 to 10 wt. %.

Steareth polymers are polyethylene glycol ethers of stearic acid. Steareth-20is a waxy compound and may reduce the interactive forces between molecules of different liquids, facilitating in the formation of an emulsion.

Palmitoyl tetrapeptide-7 is a synthetic peptide comprising amino acids glycine-glumatine-proline-arginine which are attached to the fatty acid palmitic acid to increase oil solubility and skin penetration.

The DHEA-like component may be present in the dermatological composition of the invention in the range of from 0.0005 to 12 wt. % of the total composition, preferably from 0.001 to 8 wt. %, more preferably from 0.05 to 5 wt. %.

It is an object of the present invention to provide a composition that imparts a lifting effect when applied to the skin of a user. To achieve this effect, the Kiara molecule complex comprises marine exopolysaccharides. It is believed that these components are effective at filling in fine lines and wrinkles on the skin due to their ability to induce a tightening of the collagen network of the skin by triggering the release of epidermal factors. In addition, these compounds are known to stimulate collagen, elastin and hyaluronic acid synthesis. An immediate smoothing and anti-wrinkle effect is provided as a result.

The marine exopolysaccharides preferably comprise galactose, galacturonic acid, glucose, glucuronic acid, mannose, or combinations thereof. Preferably, the composition comprises each of these components. The marine exopolysaccharides may be derived from alteromonas ferment extract. Marine beta glucans may aid in anti-aging and repairing virtues by reinforcing natural protection systems with stimulation of natural immunity cells. Phenylethyl alcohol may be included alongside alteromonas ferment extract as a masking agent.

As humans age, the process of cell regeneration slows down, contributing to the build up of dead skin cells that leaves the skin thick, dull, rough and dry. Additionally, the excess dead skin cells may clog pores, leading to break outs. To combat these undesirable effects, in addition the Kiara molecule complex, the dermatological composition of the invention may optionally comprise components that provide an exfoliating and resurfacing effect by facilitating the removal of dead skin cells. This minimises the clogging of pores and rejuvenates the skin by exposing newer, fresher skin cells. As a result, breakouts are reduced, collagen production increases, fine lines are softened, dryness and flakiness are reduced and skin appears smoother with a more even complexion. Suitable components that impart this resurfacing effect include phytic acid, alpha hydroxyl acids (AHAs) or combinations thereof.

Phytic acid may be derived from various plant sources including corn, wheat, rice, soybean and oat. This component has been found to yield gentle keratolytic effects which aids cell turnover and regeneration. It may be present in the dermatological composition in the range of from 0.001 to 5 wt. % of the total composition, preferably from 0.005 to 2 wt. %, more preferably from 0.01 to 1 wt. %.

Alpha hydroxyl acids area class of chemical compounds that comprise a carboxylic acid substituted with a hydroxyl group on the carbon at the alpha position, and may be naturally occurring or synthetic. Without wishing to be bound by theory, it is believed that AHAs are capable of sequestering calcium ions and the associated disruption of divalent metal cation-dependent cell adhesion molecules. Additionally, it is believed that they may also perform as intermediates in a variety of metabolic pathways.

Human skin has two principal components - the avascular epidermis and the underlying vascular dermis. For topical dermatological treatments, it is beneficial for some active ingredients to penetrate the epidermal layer and reach living cells within the dermis underneath.

The at least one alpha hydroxy acid (AHA) may be lactic acid, glycolic acid, citric acid, malic acid and tartaric acid. They may be derived from vaccinium myrtillus fruit extract, saccharaum officinarum extract, citrus aurantium dulcis extract, citrus lemon fruit extract, acer saccharaum extract or combinations thereof.

Glycolic acid is particularly preferred AHA. It has the smallest size of all of the AHAs which aids in its ability to penetrate the outer layer of the skin and reach deeper layers to provide immediate skin softening effects. Glycolic acid loosens corneocyte attachments and reduces cell cohesion at the lower level of the epidermis to promote cellular renewal. As new skin cells regenerate, older cells shed to reveal fresher, smoother skin. Furthermore, glycolic acid allows the capillaries to dilate and deliver increased oxygen and energy to the cells, resulting in a boost of collagen and glucosaminoglycan (GAG) synthesis. Glycolic acid treatments also increase type I collagen mRNA and hyaluronic acid content of human skin.

Lactic acid is an effective moisturising ingredient, and thus provides hydrating effects in addition to resurfacing effects. This compound is also capable of reversing cutaneous atrophy, thickening skin to create a more youthful aesthetic.

Citric acid performs as an intermediate in the Krebs cycle and is integral for ATP production, which aids in the maintenance of healthy skin by stimulating the formation of fibrous protein. The Krebs cycle is also known as the citric acid cycle, and is responsible for metabolising glucose into carbon dioxide, water and energy in the mitochondria of cells. Citric acid provides skin cells with a source of useable energy, which in turn contributes to the chemical exfoliation of the outer layer of the epidermis.

Malic and tartaric acids are AHAs which also influence skin tone and have been shown to improve skin elasticity. As a result, the strength and vitality of the skin enhances, resulting in more youthful complexion.

Preferably, the dermatological composition comprises at least one AHA. More preferably, the composition comprises at least two AHAs, more preferably at least three AHAs, more preferably at least four AHAs.

AHAs may be present in the dermatological composition in the range of from 0.0001 to 5 wt. % of the total composition, preferably from 0.005 to 2 wt. %, more preferably from 0.01 to 1 wt. %.

The composition may optionally comprise hyaluronic acid, haematococcus pluvialis extract, or combinations thereof.

Hyaluronic acid may be present in the dermatological composition, in high molecular weight (HMW) form, low molecular weight (LMW) form, or double form (HMW and LMW hyaluronic acid). It is capable of filling in lines and wrinkles to reveal a smooth complexion, while balancing the skin’s hydration and enhancing collagen synthesis.

Haematococcus extract provides natural richness in biotine, astaxanthin and B2 and C vitamins with strong anti-oxidant virtues.

The composition may comprise lactobacillus ferment lysate, camellia sinensis leaf extract, punica granatum extract, caffeine or combinations thereof. This combination was found to reduce the appearance of aging around the eyes, combatting the appearance of wrinkles, eye bags and dark circles while toning the skin for a more rejuvenated appearance.

The dermatological composition may comprise UV filters and/or UV absorbers, which work in synergy with the whitening component of the Kiara molecule to provide a skin lightening effect, and may protect against UVA and/or UVB radiation. Suitable UV filters include diethylamino hydroxybenzoyl hexyl benzoate, ethylhexyl methoxycinnamate and ehtylhexyl triazone (octyl triazone) and bis-ethylhexyylphenol methoxyphenyl triazine (bemotrizinol),

Apple blossoms stem cells may be included in the dermatological composition alongside the Kiara molecule complex. They provide anti-oxidant protection against daily external aggression, and slow the production of free radicals due to pollution. As such, they are particularly effective for protecting the skin in urban areas with high levels of pollution.

Baoab pulp may also be included in the dermatological composition, to provide an immediate tensor and lifting effect.

The dermatological composition may comprise components which impart moisturising and radiance effects to the skin. Suitable components include chondrus crispus gel extract, which has film-forming properties and soothing effects on the skin. In addition, it acts like a “vegetable silicon” and reinforces the cutaneous barrier against dehydration. Another suitable component is glucose-xylitol complex, which stimulates ceramides and glycol-amino-glycans synthesis which imparts a long lasting hydration effect. Another suitable component is a fructose-galactose sugar complex, combining fructose, galactose and galacturonic acid. It provides immediate and prolonged hydration, with the moisturising effect peaking 3 hours after application, it reduces skin irritation and gives a silky effect to the formula.

The composition may comprise thickening agents that may enhance the viscosity, volume, consistency or stability of the composition. When applied to the skin of a user, the thickening agent may also increase the ability of the skin to retain water, therefore acting as a moisturiser. Thickening agents may be natural, synthetic or semisynthetic and may include oils, waxes and fatty acids, polysaccharides, proteins, alcohols or silicones.

Optionally, the skincare composition comprises fragrance components to achieve a pleasant scent. Suitable fragrance components include essential oils, synthetic oils and combinations thereof.

The dermatological composition may also comprise pigments to provide a soft focus effect and glow to the skin. When combined with silica microspheres, these components may provide a smooth, slippery and gentle feel to the dermatological composition. In addition, these components may aid in the absorption of oil, sebum, water and sweat, providing a matte effect finish when applied to the skin.

The composition may be formulated as a solid, liquid, emulsion or gel. Preferably, the composition is formulated as a cream, serum or gel product.

The dermatological composition preferably comprises water. Water may be present in the range of 5 to 99 wt % of the total composition, preferably 10 to 90 wt %, more preferably 40 to 80 wt. %.

In a second aspect, the present invention relates to a method of dermatological treatment using the Kiara molecule. The treatment involves providing a composition comprising niacinamide, arbutin, tranexamic acid, ubiquinone, glycerine, a steareth polymer, palmitoyl tetrapeptide-7, alteromonas ferment extract and at least one uronic acid, monosaccharide or combinations thereof. The composition is then topically administered to the skin of a user.

Topical administration may involve massaging the composition into the skin of a user. This promotes the penetration of the active ingredients in the composition to the skin, and may also stimulate circulation of the skin. Massaging the product into the skin may be achieved by using fingers, a brush, and applicator device, a cotton pad, or any other suitable applicator.

The dermatological composition may be applied to the skin of a user in any desired frequency. Preferably, the composition is applied to the skin of a user twice per day. Preferably, the composition is applied in the morning and in the evening.

Preferably, the skin is cleansed prior to topical administration in order to remove dirt, oils, makeup or residue from the skin, and aid in the shedding of dead skin cells which may block the absorption of the active ingredients.

Any area of skin may be treated by the Kiara molecule, however, it is particularly effective to use on the face. The composition may be applied to the skin around the eye area, which is thinner and more delicate than the rest of the skin on the face, and is thus most susceptible to developing fine lines and wrinkles.

Therefore, the Kiara molecule is preferably administered to the skin around the eye area, including over any eye bags or dark circles, along the brow bone and over any fine lines and wrinkles around the eye area commonly known as crow’s feet.

The method of dermatological treatment may be used to treat hyperpigmentation. This may be caused by general aging of the skin, or by various conditions such as Addison’s disease, Cushing’s disease, acanthosis nigricans, melasma, acne scarring, linea nigra, Peutz-Jeghers syndrome, smoker’s melanosis, coeliac disease, Cronkhite-Canada syndrome, porphyria, tinea fungal infections such as ringworm, haemochromatosis, aromatase deficiency, Nelson’s syndrome, Grave’s disease, Schimke immunoosseous dysplasia and tinea cruris. In addition, hyperpigmentation may also be the result of exposure to certain chemicals such as salicylic acid, bleomycin, cisplatin or mercury.

It is envisaged that the Kiara molecule may therefore be used in a treatment to combat the hyperpigmentation, which may be caused by any of the above conditions.

### EXAMPLES

### Example 1

A dermatological composition according to Table 1 was formulated.

TABLE 1

| Component | Weight percentage |
|---|---|
| Water | 51.1 |
| UV filters (diethylamino hydroxybenzoyl hexyl benzoate, ethylhexyl methoxycinnamate, ethylhexyl triazone, bis-ethylhexyloxyphenol methoxyphenyl triazine) | 15.9 |
| Niacinamide | 3 |
| Marine exopolysaccharides (comprising alteromonas ferment extract and phenylethyl alcohol) | 2 |
| Tranexamic acid | 1 |
| DHEA-like component (comprising glycerine, steareth-20, chlorhexidine diglutonate, potassium sorbateand palmitoyl tetrapeptide-7) | 0.5 |
| Ubiquinone | 0.2 |
| Lecithin | 0.1 |
| Curcuma oily extract (comprising curcuma long root (turmeric) extract and helianthus annus (sunflower) seed oil) | 0.5 |
| Glucose | 0.03 |
| Arbutin liposomes | 0.02 |
| Tocopheryl acetate | 0.005 |
| Linolenic acid | 0.003 |
| Glutathione | 0.002 |
| Lactic acid | 0.001 |
| Ascorbyl palmitate | 0.0002 |
| Other components such as additives, fragrances, thickeners, stabilisers | 27.8 |
| Total | 100 |

The safety of use and the cosmetic acceptability of the product was evaluated by testing the product on 20 female volunteers. The subjects were 40-65 years old, had dark spots and wrinkles, had a variety of face skin types, had phototypes I to IV, no skin problems that could affect dermatological evaluation and no eye problems that could affect ophthamological evaluation.

The subjects applied the product twice per day in the morning and evening for a total of 6 weeks. The subjects applied the composition by massaging the product into their skin with their fingers. Specifically, the composition was applied to the eye contour area, on eye bags, eye circles, the brow bone and over crow’s feet on the face of the subject.

After the 6 week period, the tolerability and safety of the product was assessed. A dermatologist evaluated the occurrence of the following adverse skin reactions due to product use: oedema, erythema, dryness, desquamation, itching, burning, tingling and tightness. An ophthalmologist examined if the daily use of the product caused any of the following side effects: alteration of the conjunctiva, oedema, chemosis, conjunctival hyperaemia, eyelids erythema, lacrimation, xerosis, irritation sensation, itching, photophobia and foreign body sensation.

The cosmetic efficacy of the dermatological composition was also assessed by the subjects of the study by means of a questionnaire. The subjects were presented with a series of statements and questions relating to the cosmetic effects of the product and had to answer the questions with: agree, rather agree, rather disagree, disagree, or not applicable. Answers which fell into the agree and rather agree categories were considered as positive answers.

The results of the safety and tolerability test showed that no adverse skin reactions and no serious ophthalmological intolerances were found. The dermatological composition was therefore determined to be well tolerated and safe to use under the test conditions.

The results of the cosmetic efficacy questionnaire are summarised in Table 2.

TABLE 2

| Question | Positive Answers (% of subjects) |
|---|---|
| The texture of the product is pleasant | 100 |
| The product penetrates easily | 95 |
| The product does not leave the skin oily | 100 |
| The product leaves the skin instantly brighter | 67 |
| The product revives the radiance of the skin instantly | 71 |
| The product helps reduce the appearance of fine lines and wrinkles | 62 |
| The product visibly reduced the appearance of eye bags | 56 |
| The product leaves the complexion of the skin around the eyes more uniform | 76 |
| The product visibly improved the tired appearance of the skin around the eyes | 52 |
| The product leaves the skin soft and smooth | 95 |
| The product decreases the appearance of pigment and dark spots | 62 |
| The product leaves the skin hydrated | 90 |
| The product leaves the skin lighter | 67 |
| Skin looks revitalised | 67 |
| The product leaves the skin brighter | 62 |
| The product leaves the skin looking healthier | 71 |
| The product provides a lift effect | 57 |

It can be confirmed by the results of the questionnaire that the dermatologica composition provides skin lightening, brightening, lifting and smoothing effects when applied to the eye area of a user.

### Example 2

A dermatological composition according to Table 3 was formulated as a serum.

TABLE 3

| Component | Weight percentage |
|---|---|
| Water | 77 |
| Glycerin | 5 |
| DHEA-like component (comprising glycerin, steareth-20, chlorhexidine diglutonate, potassium sorbate and palmitoyl tetrapeptide-7) | 3 |
| Niacinamide | 3 |
| Marine exopolysaccharides (comprising alteromonas ferment extract and phenylethyl alcohol) | 2 |
| Tranexamic acid | 1.9 |
| Curcuma oily extract (comprising curcuma longa root (turmeric) extract and helianthus annus (sunflower) seed oil) | 0.5 |
| Ascorbyl glucoside | 0.5 |
| Camellia sinensis leaf extract | 0.5 |
| Lactobacillus ferment lysate filtrate | 0.5 |
| Punica granatum extract | 0.5 |
| Lecithin | 0.3 |
| Ubiquinone | 0.2 |
| Vaccinium myrtillus fruit extract | 0.1 |
| Lactobacillys ferment | 0.1 |
| Malus domestica fruit extract | 0.1 |
| Saccharum officinarum (sugar cane) extract | 0.06 |
| Phytic acid | 0.05 |
| Caffeine | 0.05 |
| Arbutin liposomes | 0.05 |
| Glucose | 0.03 |
| Citrus aurantium dulcis (orange) fruit extract | 0.02 |
| Tocopheryl acetate | 0.01 |
| Acer saccharum (sugar maple) extract | 0.008 |
| Linolenic acid | 0.006 |
| Glutathione | 0.005 |
| Citric acid | 0.001 |
| Lactic acid | 0.0008 |
| Ascorbyl palmitate | 0.0005 |
| Other components such as additives, fragrances, thickeners, stabilisers | 4.5 |
| Total | 100 |

The safety, tolerability and cosmetic efficacy of the product was evaluated the same method disclosed in Example 1.

The results of the safety and tolerability test showed that no adverse skin reactions and no serious ophthalmological intolerances were found. The serum was therefore determined to be well tolerated and safe to use under the test conditions

The results of the cosmetic efficacy questionnaire are summarised in Table

TABLE 4

| Question | Positive Answers (% of subjects) |
|---|---|
| The texture of the product is pleasant | 100 |
| The product penetrates easily | 86 |
| The product does not leave the skin oily | 95 |
| The product revives the radiance of the skin instantly | 62 |
| The product visibly reduced the appearance of eye bags | 67 |
| The product visibly reduced the appearance of dark circles | 52 |
| The product leaves the complexion of the skin around the eyes more uniform | 86 |
| The product visibly improved the tired appearance of the skin around the eyes | 76 |
| The product leaves the skin soft and smooth | 95 |
| The product leaves the skin hydrated | 90 |
| The product leaves the skin lighter | 62 |
| Skin looks revitalised | 57 |
| The product leaves the skin brighter | 71 |
| The product leaves the skin looking healthier | 76 |
| The product provides a lift effect | 52 |

It can be confirmed by the results of the questionnaire that the dermatological composition provides skin lightening, brightening, lifting and smoothing effects when applied to the eye area of a user.

### Example 3

The effects of the topical eye serum of Example 2 in addressing periorbital hyperpigmentation was assessed. A single-center retrospective study involving 53 patients (both male and female) was carried out to evaluate the efficacy and tolerability of the eye serum.

The serum was applied topically to the under eye area of each patient twice daily for 6 weeks.

Photographs were taken on the Aram Huvis API 100 camera of the infraorbital area of both the right eye and left eye of each patient at week 0 and at week 6. Using the Aram Huvis scoring system, the melanin was measured based on the darkness and width of melanin on the infraorbital area, and the percentage reduction in hyperpigmentation from week 0 to week 6 was established. The values represent the amount of melanin on the infraorbital area, with lower numbers representing lower levels of melanin and pigmentation, and higher numbers representing higher levels of melanin and pigmentation.

The results are shown in Table 5, and the average percentage reduction in hyperpigmentation for each eye is shown in Table 6.

TABLE 5

| Participant | Right Eye Week 0 | Right Eye Week 6 | Right Eye Percentage Reduction (%) | Left Eye Week 0 | Left Eye Week 6 | Left Eye Percentage Reduction (%) |
|---|---|---|---|---|---|---|
| 1 | 34 | 12 | 64.71 | 39 | 23 | 41.03 |
| 2 | 20 | 1 | 95.00 | 43 | 12 | 72.09 |
| 3 | 88 | 23 | 73.86 | 90 | 19 | 78.89 |
| 4 | 80 | 50 | 37.50 | 90 | 50 | 44.44 |
| 5 | 1 | 1 | 0.00 | 4 | 1 | 75.00 |
| 6 | 81 | 30 | 62.96 | 90 | 64 | 28.89 |
| 7 | 41 | 1 | 97.56 | 63 | 24 | 61.90 |
| 8 | 20 | 16 | 20.00 | 51 | 19 | 62.75 |
| 9 | 54 | 13 | 75.93 | 54 | 22 | 59.26 |
| 10 | 31 | 19 | 38.71 | 55 | 32 | 41.82 |
| 11 | 90 | 44 | 51.11 | 90 | 52 | 42.22 |
| 12 | 29 | 13 | 55.17 | 53 | 17 | 67.92 |
| 13 | 36 | 20 | 44.44 | 87 | 43 | 50.57 |
| 14 | 42 | 20 | 52.38 | 90 | 63 | 30.00 |
| 15 | 75 | 39 | 48.00 | 90 | 57 | 36.67 |
| 16 | 68 | 48 | 29.41 | 83 | 60 | 27.71 |
| 17 | 45 | 21 | 53.33 | 51 | 46 | 9.80 |
| 18 | 70 | 61 | 12.86 | 90 | 67 | 25.56 |
| 19 | 90 | 21 | 76.67 | 90 | 46 | 48.89 |
| 20 | 35 | 32 | 8.57 | 52 | 59 | -13.46 |
| 21 | 80 | 58 | 27.50 | 90 | 70 | 22.22 |
| 22 | 63 | 37 | 41.27 | 90 | 58 | 35.56 |
| 23 | 58 | 29 | 50.00 | 90 | 30 | 66.67 |
| 24 | 31 | 17 | 45.16 | 90 | 42 | 53.33 |
| 25 | 47 | 38 | 19.15 | 81 | 50 | 38.27 |
| 26 | 44 | 41 | 6.82 | 74 | 51 | 31.08 |
| 27 | 90 | 34 | 62.22 | 90 | 43 | 52.22 |
| 28 | 90 | 14 | 84.44 | 90 | 17 | 81.11 |
| 29 | 90 | 67 | 25.56 | 90 | 67 | 25.56 |
| 30 | 90 | 74 | 17.78 | 78 | 65 | 16.67 |
| 31 | 90 | 75 | 16.67 | 90 | 77 | 14.44 |
| 32 | 90 | 78 | 13.33 | 73 | 65 | 10.96 |
| 33 | 32 | 10 | 68.75 | 64 | 10 | 84.38 |
| 34 | 44 | 41 | 6.82 | 46 | 41 | 10.87 |
| 35 | 90 | 41 | 54.44 | 65 | 41 | 36.92 |
| 36 | 90 | 32 | 64.44 | 90 | 32 | 64.44 |
| 37 | 90 | 50 | 44.44 | 84 | 50 | 40.48 |
| 38 | 81 | 50 | 38.27 | 77 | 50 | 35.06 |
| 39 | 35 | 10 | 71.43 | 35 | 18 | 48.57 |
| 40 | 90 | 64 | 28.89 | 90 | 64 | 28.89 |
| 41 | 81 | 37 | 54.32 | 81 | 37 | 54.32 |
| 42 | 59 | 13 | 77.97 | 59 | 13 | 77.97 |
| 43 | 69 | 9 | 86.96 | 72 | 32 | 55.56 |
| 44 | 80 | 13 | 83.75 | 80 | 13 | 83.75 |
| 45 | 80 | 14 | 82.50 | 80 | 20 | 75.00 |
| 46 | 90 | 23 | 74.44 | 90 | 23 | 74.44 |
| 47 | 90 | 49 | 45.56 | 90 | 49 | 45.56 |
| 48 | 11 | 2 | 81.82 | 83 | 11 | 86.75 |
| 49 | 47 | 15 | 68.09 | 52 | 45 | 13.46 |
| 50 | 31 | 29 | 6.45 | 90 | 30 | 66.67 |
| 51 | 84 | 40 | 52.38 | 70 | 32 | 54.29 |
| 52 | 20 | 24 | –20.00 | 48 | 25 | 47.92 |
| 53 | 40 | 5 | 87.50 | 62 | 7 | 88.71 |

TABLE 6

| | |
|---|---|
| Average Right Eye Percentage Reduction (%) | 48.44 |
| Average Left Eye Percentage Reduction (%) | 47.43 |
| Average Overall Reduction (%) | 47.94 |

The results confirm that the daily use of the eye serum results in an average overall reduction in under eye hyperpigmentation of 47.93%.

The results demonstrate that an eye serum containing a blend of naturally derived and synthetic extracts according to the invention achieved global skin rejuvenation by significantly improving appearance of periorbital hyperpigmentation.

The test product was also highly rated by subjects on performance and ease of application, and was well tolerated by all participants in this study.

### REFERENCES

Weringhaus et al., “Evaluation of the photoprotective effect of oral vitamin E supplementation”, Archives of Dermatology 130(10), 1257-1261 (1994);

Poljsak and Dahmane, “Free Radicals and Extrinsic Skin Aging”, Dermatology Research and Practice 2012, 1-4 (2012);

Nimse and Pal, “Free radicals, natural antioxidants, and their reaction mechanisms” RSC Advances 5(35), 27986-28006 (2015);

Cichorek et al., Central European Journal of Biology, “Heterogeneity of neural crest-derived melanocytes” 8(4), 315-330 (2013);

Rea et al., “Age and Age-Related Diseases Role of Inflammation Triggers and Cytokines”, Frontiers in Immunology 9(586), 1-28 (2018).

1. A dermatological composition comprising niacinamide, arbutin, tranexamic acid, ubiquinone, a DHEA-like component, curcuma oily extract and marine exopolysaccharides.

2. The composition of claim 1 further comprising ascorbyl glucoside.

3. The composition of claim 1, wherein the curcuma oily extract comprises curcuma longa root extract.

4. The composition of claim 3, wherein the curcuma oily extract further comprises helianthus annus seed oil.

5. The composition of claim 1, wherein the DHEA-like component comprises glycerin, steareth-20 and palmitoyl tetrapeptide-7 and preferably chlorhexidine digluconate and potassium sorbate.

6. The composition of claim 1, wherein the marine exopolysaccharides comprises galactose, galacturonic acid, glucose, glucuronic acid, mannose or combinations thereof.

7. The composition of claim 1, wherein the marine exopolysaccharides are derived from alteromonas ferment extract.

8. The composition of claim 1 further comprising at least one alpha hydroxy acid.

9. The composition of claim 1, wherein the alpha hydroxy acid is derived from vaccinium myrtillus fruit extract, saccharaum officinarum extract, citrus aurantium dulcis extract, citrus lemon fruit extract, acer saccharaum extract or combinations thereof.

10. The composition of claim 1 further comprising hyaluronic acid, haematococcus pluvialis extract or combinations thereof.

11. The composition of claim 1 further comprising lactobacillus ferment lysate, camellia sinensis leaf extract, punica granatum extract, caffeine or combinations thereof.

12. The composition of claim 1, wherein the composition is formulated as a cream, serum or gel product.

13. A method of dermatological treatment comprising:
a) providing a composition comprising niacinamide, arbutin, tranexamic acid, ubiquinone, a DHEA-like component, curcuma oily extract and marine exopolysaccharides; and
b) topically administering the composition to the skin of a user.

14. The method of claim 13, wherein the composition comprises ascorbyl glucoside.

15. The method of claim 13, wherein the method further comprises massaging the composition into the skin of a user until the composition is absorbed by the skin.

16. The method of claim 13, wherein the composition is applied to the skin of a user twice per day.

17. The method of claim 13, wherein the composition is applied to the skin around the eye area.

18. A method of treating hyperpigmentation comprising:
a) providing a composition comprising niacinamide, arbutin, tranexamic acid, ubiquinone, a DHEA-like component, curcuma oily extract and marine exopolysaccharides; and
b) topically administering the composition to the skin of a user.

19. The method of claim 18, wherein the composition comprises ascorbyl glucoside.
